(19) United States
(12) Patent Application Publication
(10) Pub. No.: US 2009/0011046 A1
(43) Pub. Date: Jan. 8, 2009

(54) Composition Comprising Silver Nanoparticles and the Use Thereof

(76) Inventor: Chenlung Lin, Hong Kong (CN)

Correspondence Address: BEYER WEAVER LLP, P.O. BOX 70250, OAKLAND, CA, 94612-0250, US

(21) Appl. No.: 11/997,726

(22) PCT Filed: Aug. 4, 2005
(86) PCT No.: PCT/CN2005/001197

§ 371 Date: Jul. 10, 2008

(51) Int. Cl. A61K 33/38; A61Q 7/00

(52) U.S. Cl. 424/618

(57) ABSTRACT

The present invention relates to a pharmaceutical composition comprising an effective amount of silver nanoparticles, a method for increasing hair growth by administering the said pharmaceutical composition locally to a host and use thereof.

### FIELD OF THE INVENTION

The present invention relates to a pharmaceutical composition comprising an effective amount of silver nanoparticles, a method for increasing hair growth by administering the said pharmaceutical composition locally to a host and use thereof.

### BACKGROUND OF THE INVENTION

Metal nanoparticles have been attracting increasing attention to the chemical society due to their important applications in a number of subject areas such as catalysis and nanoscale electronics (references: (a) El-Sayed, M. A. Acc. Chem. Res. 2004, 37, 326. (b) Ho, C.-M.; Yu, W.-Y.; Che, C.-M. Angew. Chem. Int. Ed. 2004, 43, 3303. (c) Lang, H.; May, R. A.; Iversen, B. L.; Chandler, B. D. J. Am. Chem. Soc. 2003, 125, 14832. (d) Lewis, L. N. Chem. Rev. 1993, 93, 2693). Recently, much effort has been devoted to develop biomedical applications of metal nanoparticles. While significant advance in biological labeling has been made (references: (a) Nicewarner-Pena, S. R.; Freeman, R. G.; Reiss, B. D.; He, L.; Pena, D. J.; Walton, I. D.; Cromer, R.; Keating, C. D.; Natan, M. J. Science 2001, 294, 137. (b) Elghanian, R.; Storhoff, J. J.; Mucic, R. C.; Letsinger, R. L.; Mirkin, C. A. Science 1997, 277, 1078), few therapeutic applications of metal nanoparticles have been reported in the literature. A notable example is the anti-microbial properties of silver nanoparticles, which has been used for wound healing (Wright, J. B.; Lam, K.; Hansen, D.; Burrell, R. E. Am. J. Inf. Cont. 1999, 27, 344). To our knowledge, the use of silver nanoparticles to increase hair growth in alopecia has not been found in the literature.

Alopecia areata is a condition in which there is hair loss on the scalp and elsewhere on the body. It has been reported to be associated with autoimmunity by which the hair follicles become attached by self immune system, resulting in an arrest of the hair growth. Another observation is that high levels of a naturally occurring hormone called dihydroxytestosteron (DHT) are present in the scalp of men genetically disposed to male pattern hair loss, the growth phase of hair is shortened. Eventually, the hairs become so small as to be practically invisible. This can result in the classic male bald hairline if it is not stopped.

Alopecia areata usually starts with one or more small, round, smooth bald patches on the scalp and can progress to total scalp hair loss (alopecia totalis) or complete body hair loss (alopecia universalis). Epidemiology-wise, alopecia areata affects approximately 1.7 percent of the population all over the world, including more than 4.7 million people in the United States alone, and this condition/disease has a profound impact on one's self-image and self-confidence, thereby affecting the life quality, both at work and at school.

Alopecia areata can occur in males and females of all ages and races. Its onset most often begins in childhood/early adulthood and can be psychologically devastating. Although alopecia areata is not life-threatening, it is most certainly life-altering, and its sudden onset, recurrent episodes, and unpredictable course have a profound psychological impact on the lives of those disrupted by this disease.

### CONTENTS OF THE INVENTION

The inventor of the present application found that by using human serum albumin (HAS) as a stabilizer for the fabrication of silver nanoparticles, these nanoparticles have demonstrated promising effects in increasing hair growth in an animal model and humans.

Therefore, the present invention relates to a pharmaceutical composition comprising an effective amount of silver nanoparticles, at least one stabilizer, for examples one or more proteins and/or peptide, and at least one pharmaceutically acceptable diluent. Preferably, the protein is selected from the group consisting of human serum albumin and transferrin.

In a preferred embodiment, the pharmaceutical composition of the present application comprises a diluent selected from the group consisting of water, ethanol, DMSO, acetonitrile, Hepes buffer, phosphate buffer, Tris buffer, citrate buffer, serum or any other kinds of physiologically relevant solvent or solution, and a mixture thereof.

The present invention also relates to a method for increasing hair growth comprising administering locally the pharmaceutical composition of the present invention to a host, preferably human, needed to be treated.

The present invention also relates to use of a composition comprising an effective amount of silver nanoparticles in manufacture of a medicament for increasing hair growth in a host.

In the use of the present invention, the composition comprises an effective amount of silver nanoparticles, at least one stabilizer, for examples one or more proteins and/or peptide, and at least one pharmaceutically acceptable diluent. Preferably, the protein is selected from the group consisting human serum albumin and transferrin.

In a preferred embodiment of use, the pharmaceutical composition of the present application comprises a diluent selected from the group consisting of water, ethanol, DMSO, acetonitrile, Hepes buffer, phosphate buffer, Tris buffer, citrate buffer, serum or any other kinds of physiologically relevant solvent and solution, or a mixture thereof.

There are numerous methods for generating silver nanoparticles with different shapes and sizes (references: (a) Raveendran, P.; Fu, J.; Wallen, S. L. J. Am. Chem. Soc. 2003, 125, 13940. (b) Sun, Y.; Xia, Y. Science 2002, 298, 2176. (c) Esumi, K.; Suzuki, A.; Yamahira, A.; Torigoe, K. Langmuir 2000, 16, 2604). In the present invention, the silver nanoparticles was readily prepared using Hepes buffer. A typical reaction was by dissolving AgNO₃ (2 or 5 mM) in Hepes buffer (0.1 M, pH 7.4) to a final concentration of 1 mM AgNO₃ at room temperature and stirring at room temperature for 120 h or stirring under refluxing condition for 4 h. Formation of the nanoparticles was affected by buffer acidity, since no silver nanoparticles was formed when the pH was adjusted to <5. From FIG. 1, it can be seen that the nanoparticles prepared are uniformly distributed essentially with particle size varied from 5 to 20 nm (with average diameter about 10 nm). A spot-profile energy-dispersive X-ray analysis (EDX) showed the presence of strong signals from the silver atoms together with Cu atom signal from the copper grid and weaker signals that are due to C, O and Cl atoms (FIG. 2).

The powder X-ray diffraction (XRD) pattern taken from a larger quantity of sample suggested that the silver nanoparticles generated in Hepes buffer existed in face-center-cubic phase. As depicted in FIG. 3, the presence of intense peaks corresponding to 20 values of (111), (200), (220) and (311) in the powder X-ray diffraction pattern agrees with those values reported in literature (JCPDS No. 04-0783). The silver nanoparticles exhibit an absorption peak at 410 nm (FIG. 4), which is close to the reported data in the powder X-ray diffraction (XRD) pattern taken from a larger quantity of sample.

Based on UV-Vis spectroscopy, in the presence of HSA, no significant spectral change of the silver nanoparticles with concentration up to 1 mM was observed at 37° C. for even up to 7 days. In this sample, HSA (1 mM, at physiologically relevant level) was employed to stabilize silver nanoparticles (see FIG. 5).

From the UV-vis absorption titration (see FIG. 6), it can be seen that the silver nanoparticles may interact with HSA (Δλₘₐₓ=+4 nm; hyperchromicity 3%).

TEM images showed that there was a change in size from 10 nm to 20 nm when the silver nanoparticles were dissolved in 1 mM HSA for 3 days; nevertheless, no silver precipitation was observed (see FIG. 7).

The effect of the silver nanoparticles on hair growth was studied by using an animal model. In this animal model, nude mice were applied with gauze dressing at the posterior scalp with 1 mM reference control (i.e., human serum albumin; HSA) only or 0.1 mM silver nanoparticle in 1 mM HSA. After 42 days, hair growth could be observed at the posterior scalp of mice applied with silver nanoparticle (see right panel of FIG. 8) but not in mice with reference control alone (see left panel of FIG. 8). n=5 for each group.

In another study, two groups of nude mice were applied with 1 mM reference control or 1 mM silver nanoparticles in 1 mM HSA in the same manner as above at the posterior scalp. After 42 days, hair growth could be observed at the posterior scalp applied with silver nanoparticles (right panel) but not in mice with reference control alone (left panel). n=5 for each group (see FIG. 9).

Histology of the full-thickness skin was taken from mice (Balb/c) with normal hair growth, from nude mice applied with reference control only or with 1 mM silver nanoparticles for 42 days. Results indicate that skin from nude mice applied with silver nanoparticles had normal-looking hair follicles at the dermis (see middle panel of FIG. 10), at least by morphology, similar to that found in mice with normal hair growth (see upper panel of FIG. 10). In contrast, skin taken from nude mice applied with reference control had few identifiable hair follicles (see lower panel of FIG. 10).

Stability is an important issue of silver nanoparticles for therapeutic applications. Previous studies showed that silver nanoparticles were unstable in solution and would easily aggregate at high concentration or with average particle size>40 nm (reference: Mafune, F.; Kohno, J.-Y.; Takeda, Y.; Kondow, T.; Sawabe, H. J. Phy. Chem. B 2000, 104, 8333). To maintain adequate solution stability, the size of the silver nanoparticles prepared in this study were confined to 5 to 20 nm in diameter. These nanoparticles exhibited excellent stability at 50 μM in Hepes buffer; no significant spectral change was observed over 3 days at 37° C. based on UV-vis spectroscopy. However, at 1 mM level, silver precipitation in commitment with UV spectral changes were found after 3 days incubation at 37° C. Similarly, silver nanoparticles prepared in citrate buffer by using traditional NaBH₄ protocol also demonstrated good solution solubility at 50 μM, but poor solution stability at 1 mM level.

Human serum albumin (HSA) is the most abundant plasma protein in circulatory system. Previous reports showed that this serum protein could be used to stabilize a variety of metal nanoparticles (reference: Xie, H.; Tkachenko, A. G.; Glomm, W. R.; Ryan, J. A.; Brennaman, M. K.; Papanikolas, J. M.; Franzen, S.; Feldheim, D. L. Anal. Chem. 2003, 75, 5797). In this study, HSA (1 mM, at physiologically relevant level) was employed to stabilize silver nanoparticles. Based on UV-Vis spectroscopy, we found that in the presence of HSA, no significant spectral change of the silver nanoparticles with concentration up to 1 mM was observed at 37° C. for even up to 7 days (FIG. 5). Recently, Shen and co-workers had demonstrated hysteresis effect of the interaction between serum albumins and silver nanoparticles (60 nm) (Shen, X.; Yuan, Q.; Liang, H.; Yan, H.; He, X. Sci. in China 2003, 46, 387). By means of UV-Vis absorption titration, we also found that the silver nanoparticles would interact with HSA (Δλₘₐₓ=+4 nm; hyperchromicity=3%, FIG. 6). TEM images showed that there was a change in size from 10 nm to ˜20 nm when the silver nanoparticles were treated with HSA for 3 days; nevertheless, no silver precipitation was observed (see FIG. 7).

### BRIEF DESCRIPTION OF FIGURES

FIG. 1 shows transmission electron microscopy (TEM, A and B) and high-resolution transmission electron microscopy (HR-TEM, C and D) images of the silver nanoparticles prepared in Example 1.

FIG. 2 is the Spot-profile energy-dispersive X-ray analysis (EDX) of the silver nanoparticles prepared in Example 1.

FIG. 3 is the powder X-ray diffraction (XRD) pattern taken from a larger quantity of sample prepared in Example 1.

FIG. 4 is the absorption spectra of the silver nanoparticles prepared in Example 1.

FIG. 5 is the UV-Vis spectroscopies of the 1 mM silver nanoparticles in the presence of 1 mM HSA observed at 37° C. for 7 days.

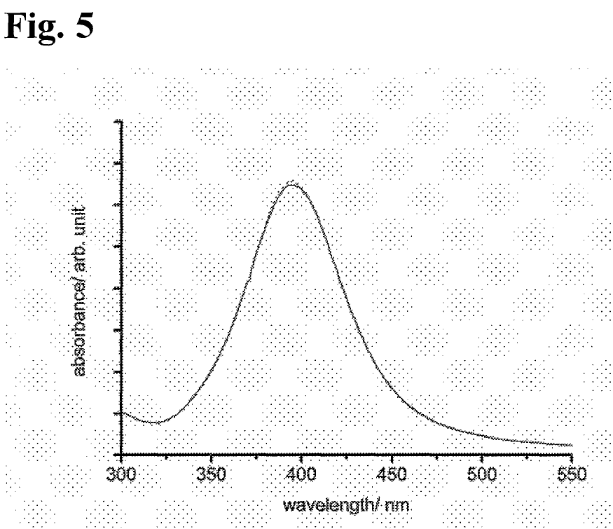

FIG. 6 shows UV-vis absorption titration of the silver nanoparticles with HSA.

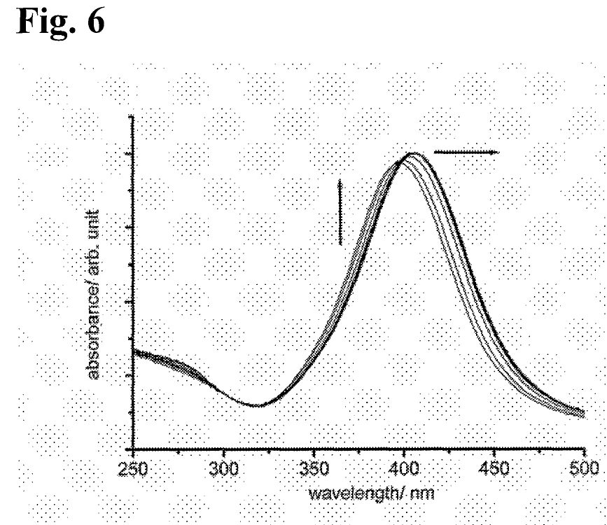

FIG. 7 is the transmission electron microscopy images of the silver nanoparticles dissolved in 1 mM HSA for 3 days.

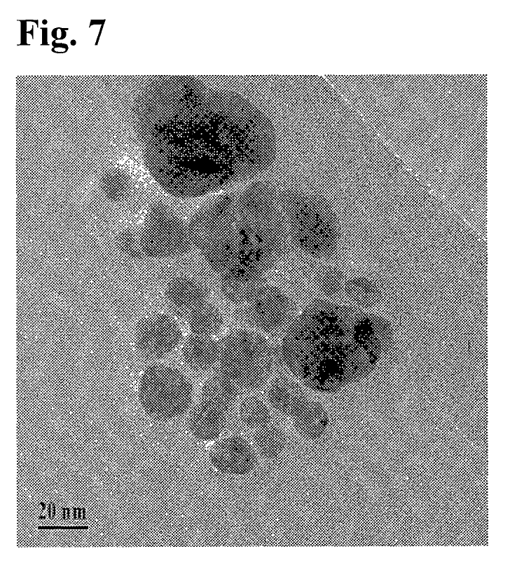

FIG. 8 are the photos of nude mice 42 days after daily applied with gauze dressing at the posterior scalp with 1 mM reference control (i.e., human serum albumin; HSA) only (left panel) or with 0.1 mM silver nanoparticle in 1 mM HAS (right panel). n=5 for each group.

FIG. 9 are the photos of nude mice 42 days after daily applied with gauze dressing at the posterior scalp with 1 mM reference control (i.e., human serum albumin; HSA) only (left panel) or with 1 mM silver nanoparticle in 1 mM HAS (right panel). n=5 for each group. n=5 for each group.

FIG. 10 shows the histology of the full-thickness skin taken from mice (Balb/c) with normal hair growth (upper panel), from nude mice applied with reference control only (lower panel) or with silver nanoparticles for 42 days (middle panel).

### MODE OF CARRYING OUT THE INVENTION

The present invention will now be further explained in the following examples. However, the present invention should not be construed as limited thereby. One of ordinary skill in the art will understand how to vary the exemplified preparations to obtain the desired results.

### Examples

Materials. All chemicals, except otherwise noted, were purchased from Sigma-Aldrich Chemical Co.

Instrumentation. The XRD spectrum was recorded with Philips PW1830 powder X-ray diffractometer. UV-vis absorption spectra were taken using Varian Cary 50 spectrophotometer. TEM images of the silver nanoparticles were taken with JEOL JEM-2000 transmission electron microscopy, using an accelerating voltage of 200 kV, and were carried out on the Philips Tecnai 20 equipped with Oxford incax-sight EDX attachment using an accelerating voltage of 200 kV and Philips EM208s using an accelerating voltage of 80 kV.

Preparation of silver nanoparticles. Aqueous AgNO₃ solution (2-50 mM, 1 mL) was slowly added to the round bottom flask stirring with a magnetic stirring bar containing human serum albumin and water, to the final concentration of 1 mM of HAS and 1 mM of AgNO₃. The actual concentration of the silver nanoparticles was measured by Inductively-Coupled Plasma Mass Spectroscopy (ICP-MS).

Absorption Titration. A solution of silver nanoparticles (5 μM) in Hepes buffer (3000 μL) was placed in a thermostatic cuvette in a UV-vis spectrophotometer. Aliquots of a millimolar stock human serum album (HSA) solution were added to the solution. Absorption spectra were recorded after equilibration for 10 min per aliquot until saturation point has reached.

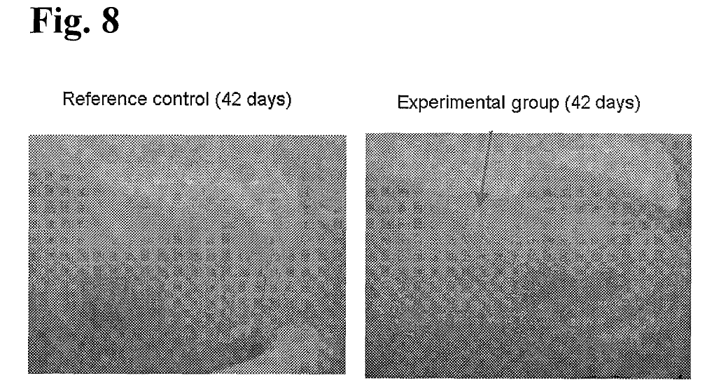

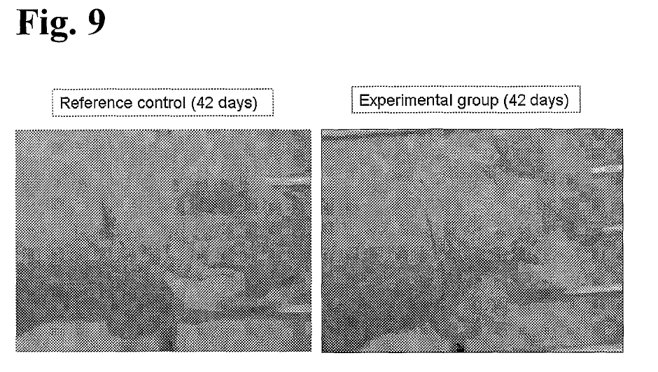

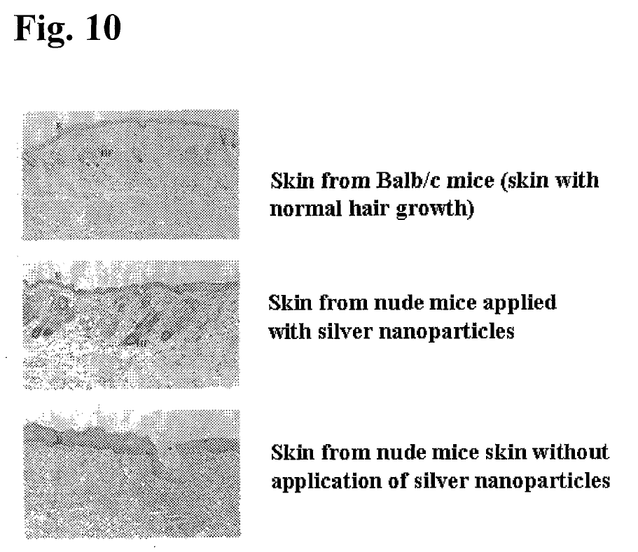

Biological Studies and Measurement. Nude mice that are with Whn (Winged-helix) mutation, were purchased from Jackson lab (Canada), and were used at 4-5 weeks old. Before experiments, these nude mice were inspected to ascertain there was no visible hair (reference: Anat Gafter-Gvili, Benjamin Sredni, Rivka Gal, Uzi Gafter, and Yona Kalechman. Cyclosporin A-induced hair growth in mice is associated with inhibition of calcineurin-dependent activation of NFAT in follicular keratinocytes Am J Physiol Cell Physiol 284:1593-1603, 2003). A 1×1 cm gauze was soaked with 500 μl of 0.1 or 1 mM silver nanoparticles in HSA solution and dressed at the posterior scalp (silver nanoparticle was used as 0.1 mM in FIG. 8) or at the posterior scalp (silver nanoparticle was used as 1 mM in FIG. 9). The dressing was changed every day until day 42. Direct inspection) was performed every day until day 42. Selective mice were sacrificed on day 42 and the skin was taken for histology for the morphology of the hair follicles (as shown in FIG. 10).

### Example One

### Preparation and Characterization of Silver Nanoparticles

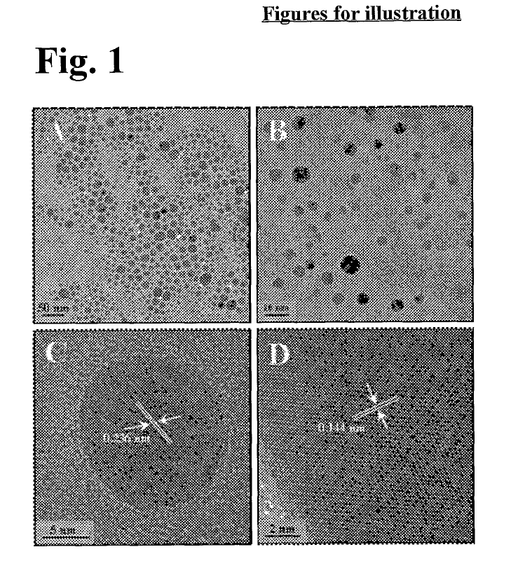

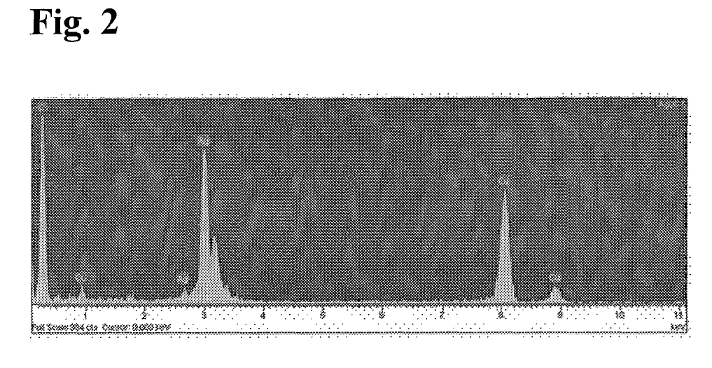

A) the silver nanoparticles were obtained by dissolving AgNO₃ (5 mM) in Hepes buffer (0.1 M, pH 7.4) at room temperature to a final concentration of 1 mM of AgNO₃ and stirring at room temperature for 120 h. FIG. 1 shows transmission electron microscopy (TEM, A and B) and high-resolution transmission electron microscopy (HR-TEM, C and D) images of the silver nanoparticles. These nanoparticles are uniformly distributed with particle size varied from 5 to 20 nm (with average diameter about 10 nm). A spot-profile energy-dispersive X-ray analysis (EDX) showed the presence of strong signals from the silver atoms together with Cu atom signal from the copper grid and weaker signals that are due to C, O and Cl atoms (FIG. 2).

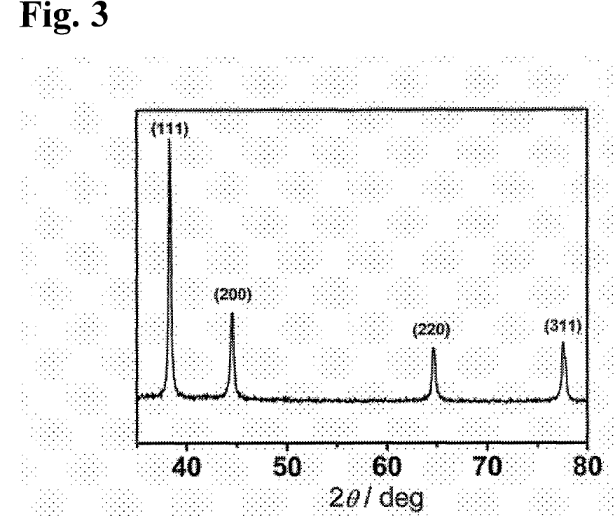

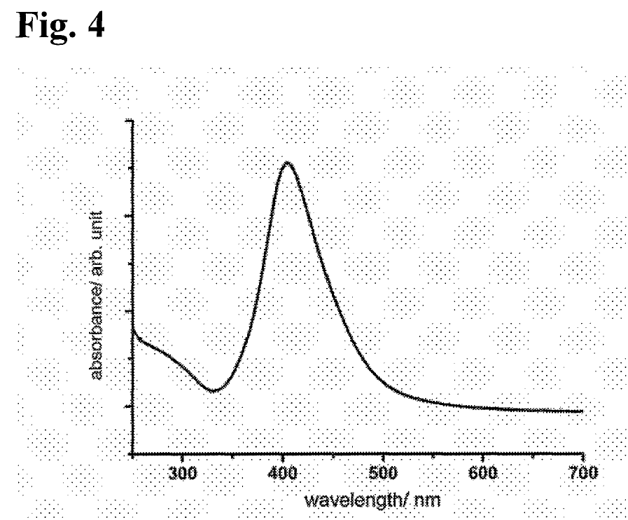

The powder X-ray diffraction (XRD) pattern taken from a larger quantity of sample suggested that the silver nanoparticles generated in Hepes buffer existed in face-center-cubic phase. As depicted in FIG. 3, the presence of intense peaks corresponding to 2θ values of (111), (200), (220) and (311) in the powder X-ray diffraction pattern agrees with those values reported in literature (JCPDS No. 04-0783). The silver nanoparticles exhibit an absorption peak at 410 nm (FIG. 4), which is close to the reported data.

B) the same results were obtained by repeating the experiment A), but using AgNO₃ (2 mM).

C) the same results were obtained by repeating the experiment A), except that the mixture was stirred under refluxing condition for 4 h instead of at room temperature for 120 h.

### Example Two

### Effect of Silver Nanoparticles in Increasing Hair Growth of Nude Mice after Application for 42 Days at the Posterior Scalp

Nude mice were applied with gauze dressing at the posterior scalp with 1 mM reference control only (i.e., human serum albumin; HSA) or 0.1 mM silver nanoparticle in 1 mM HSA. Dressing was changed daily. After 42 days, hair growth could be observed at the posterior scalp of mice applied with silver nanoparticle (right panel, FIG. 8) but not in mice with reference control alone (left panel, FIG. 8). n=5 for each group.

### Example Three

### Effect of Silver Nanoparticles in Increasing Hair Growth of Nude Mice after Application for 42 Days at the Posterior Scalp

Additional two groups of nude mice were applied with 1 mM reference control or 1 mM silver nanoparticle in 1 mM HSA in the same manner as in EXAMPLE TWO at the posterior scalp. After 42 days, hair growth could be observed at the posterior scalp applied with silver nanoparticles (right panel, FIG. 9) but not in mice with reference control alone (left panel, FIG. 9). n=5 for each group.

### Example Four

### Histology of the Skin Taken from Mice with Normal Hair Growth, from Nude Mice Applied with or without Silver Nanoparticles after 42 Days' Application As Described in Example Three

Histology of the full-thickness skin was taken from mice (Balb/c) with normal hair growth, from nude mice applied with reference control only or with 1 mM silver nanoparticles in 1 mM HSA for 42 days. Results indicate that skin from nude mice applied with silver nanoparticles had normal-looking hair follicles at the dermis (middle panel, FIG. 10), at least by morphology, similar to that found in mice with normal hair growth (upper panel, FIG. 10). In contrast, skin taken from nude mice applied with reference control had few identifiable hair follicles (lower panel, FIG. 10).

### Example Five

### Testing of the Toxicity of the Silver Nanoparticles on Mice

10 mM of silver nanoparticles in 1 mM HSA were given to normal, healthy mice per day for 42 days. This dose was more than 100 fold of the effective dose for hair growth. The activities of mice were monitored. At the end of the 42 days period, no mice were dead or sick, which indicated that the silver nanoparticles in HAS were safe and non-toxic to mice.

### Example Six

### Skin Allergic Test of the NAGs on Mice

1 mM of silver nanoparticles in 1 mM HSA was applied to the skin of the nude mice. The mice were monitored at 1 hr, 24 hrs, and 48 hrs intervals. No rash, bruise, or any irritating reaction was observed on the skin, which indicated that the silver nanoparticles in HSA were safe and not creating any allergic reaction on the skins.

1. A pharmaceutical composition comprising an effective amount of silver nanoparticles, at least one stabilizer and at least one pharmaceutically acceptable diluent.

2. The pharmaceutical composition of claim 1 wherein one or more proteins and/or peptide are used as stabilizers.

3. The pharmaceutical composition of claim 2 wherein the protein is selected from the group consisting of human serum albumin and transferrin.

4. The pharmaceutical composition of any claim 1 wherein the silver nanoparticles are 1 to 100 nm in diameter and the concentration of the silver nanoparticles in the composition is 10 μM to 10 mM.

5. The pharmaceutical composition of claim 4 wherein the concentration of the silver nanoparticles in the composition is 100 μM to 1 mM.

6. The pharmaceutical composition of claim 1 wherein the diluent is selected from the group consisting of water, ethanol, DMSO, acetonitrile, Hepes buffer, phosphate buffer, Tris buffer, citrate buffer, serum or any other kinds of physiologically relevant solvent or solution, and a mixture thereof.

7. A method for increasing hair growth comprising administering locally the pharmaceutical composition according to claim 1 to a host needed to be treated.

8. The method of claim 7 wherein the host is human.

9. Use of a composition comprising an effective amount of silver nanoparticles in manufacture of a medicament for increasing hair growth in a host.

10. Use of claim 9 wherein the composition comprises an effective amount of silver nanoparticles, at least one stabilizer and at least one pharmaceutically acceptable diluent.

11. Use of claim 10 wherein one or more proteins and/or peptide are used as stabilizers.

12. Use of claim 11 wherein the protein is selected from the group consisting of human serum albumin and transferrin.

13. Use of claim 9 wherein the silver nanoparticles are 1 to 100 nm in diameter and the concentration of the silver nanoparticles in the composition is 10 μM to 10 mM.

14. Use of claim 13 wherein the concentration of the silver nanoparticles in the composition is 100 μM to 1 mM.

15. Use of claim 9 wherein the diluent is selected from the group consisting of water, ethanol, DMSO, acetonitrile, Hepes buffer, phosphate buffer, Tris buffer, citrate buffer, serum other kinds of physiologically relevant solvent or solution, and a mixture thereof.

16. Use of claim 9 wherein the host is human.
